(19) United States
(12) Patent Application Publication
(10) Pub. No.: US 2012/0157970 A1
(43) Pub. Date: Jun. 21, 2012

(54) SPIRAL FLOW INFUSION CANNULA

(76) Inventors: Michael McCulloch Martin, (US); Brian Daniel Quinn, (US)

(21) Appl. No.: 12/973,163

(22) Filed: Dec. 20, 2010

(51) Int. Cl. A61M 25/14

(52) U.S. Cl. 604/523

(57) ABSTRACT

An infusion cannula adapted to generate a spiral, laminar flow therein. In some implementations, the infusion cannula may include one or more flow-altering devices disposed within a passageway of the cannula. The flow-altering devices may include one or more blades adapted to form a spiral flow in the laminar flow regime. In some instances, the flow altering-device may include one or more baffles. Further, in other instances, the flow altering device may include a flow tube. The flow tube may be disposed tangential to a passageway of the cannula.

### TECHNICAL FIELD

The present disclosure relates to infusion cannulas operable to deliver fluids with a spiral, laminar flow. Particularly, the present disclosure is directed to infusion cannulas operable to infuse fluids having spiral, laminar flow during ocular surgeries.

### BACKGROUND

Cannulas may be used in surgical procedures. For example, cannulas are inserted into a body cavity for infusing or withdrawing fluid therefrom. An infusion cannula may be utilized in ocular surgery to introduce or infuse fluids into the eye, such as to maintain intraocular pressure.

### SUMMARY

According to one aspect, the disclosure describes an infusion cannula having a body including a first portion defining a first bore and a second portion defining a second bore. The first bore and the second bore are in fluid communication with each other. A passageway may be defined by the first bore and the second bore. The passageway may extend through the body. The cannula may also include a flow-altering device adapted to generate a spiral, laminar flow of fluid passing through the passageway.

The various aspects may include one or more of the following features. The first bore may have a cross-sectional area that is larger than a cross-sectional area of the second bore. The first bore and the second bore may have circular cross-sections, and the first bore may have a larger cross-sectional area than the second bore. The flow-altering device may include a blade disposed at a location in the passageway. The blade may be disposed in the second bore proximate a junction between the first bore and the second bore. The blade may include a leading edge at an upstream location and a trailing edge at a downstream location, and an angle of attack of the leading edge may be less than an angle attack of the trailing edge. The blade may be coupled to an interior wall of the passageway at least one location. The blade may be coupled to the interior wall of the passageway at two locations. The blade may diametrically extend across the passageway. A plurality of blades may be disposed in the passageway. A first portion of the plurality of blades may be disposed in the first bore, and a second portion of the plurality of blades may be disposed in the second bore.

The various aspects may also include one or more of the following features. At least one blade may be radially offset from another blade. The flow-altering device may be a spiral member disposed at a location along the passageway. The spiral member may include a leading edge at an upstream location and a trailing edge at a downstream location. The leading edge may include a first angle of attack and the trailing edge having a second angle of attack. The first angle of attack may be greater than the second angle of attack. An angle of attack of the spiral member may vary from the first angle of attack to the second angle of attack. An angle of attack of the spiral member may decrease from a first angle of attack at a leading edge of the spiral member at an upstream location to a second angle of attack at a trailing edge of the spiral member at a downstream location. An angle of attack of the spiral member may increase from a first angle of attack at a leading edge of the spiral member at an upstream location to a second angle of attack at a trailing edge of the spiral member at a downstream location. The spiral member may have a constant angle of attack along a length thereof. The spiral member may extend through a portion of the first bore and through a portion of the second bore.

The details of one or more implementations of the present disclosure are set forth in the accompanying drawings and the description below. Other features, objects, and advantages will be apparent from the description and drawings, and from the claims.

### DESCRIPTION OF DRAWINGS

FIG. 1 shows a cross-sectional view of an example infusion cannula including a blade adapted to generate a spiral, laminar flow.

FIG. 2 shows an end view of the example cannula shown in FIG. 1.

FIGS. 3 and 4 are partial cross-sectional views of another example infusion cannula including baffles adapted to generate spiral, laminar flow.

FIGS. 5 and 6 are detail views of the example infusion cannula shown in FIGS. 3 and 4.

FIG. 7 shows a further example of an infusion cannula including a spiral flow-altering device.

FIG. 8 shows a detail cross-sectional view of another example infusion cannula including a spiral flow-altering feature adapted to generate a spiral, laminar flow.

FIG. 9 is a perspective view of another example infusion cannula including a flow tube.

FIGS. 10-11 show an end view and a cross-sectional view, respectively, of an example infusion cannula having a flow tube extending substantially perpendicularly to a longitudinal axis of the body of the infusion cannula.

FIGS. 12 and 13 show and end view and a cross-sectional view, respectively, of a further example infusion cannula having a flow tube extending obliquely relative to the longitudinal axis of the body of the infusion cannula.

FIGS. 14 and 15 show cross-sectional view of additional example infusion cannulae having tapered flow tubes.

FIGS. 16 and 17 show end views of further example infusion cannulae in which the flow tubes have different diameters.

### DETAILED DISCLOSURE

The present disclosure is directed to cannulas adapted to generate a spiral, laminar flow therein and expel a diffused flow of fluid from the cannulas. The cannulas described herein may be used in surgical procedures. In some implementations, the cannulas may be used in ophthalmic surgical procedures. The spiral, laminar flow generated in the cannulas provides a reduced pressure drop through the cannula compared to pressure drops associated with turbulent flows. The greater pressure drop generated by turbulent flow also causes a reduction in flow rate. Consequently, the laminar flow may also provide an increased fluid flow rate of fluid exiting the cannula. Additionally, the spiral, laminar flow does not introduce a jet of fluid exiting the cannula that could cause injury to delicate tissues within a patient. Rather, the expelled fluid flow of a spiral, laminar flow is diffuse.

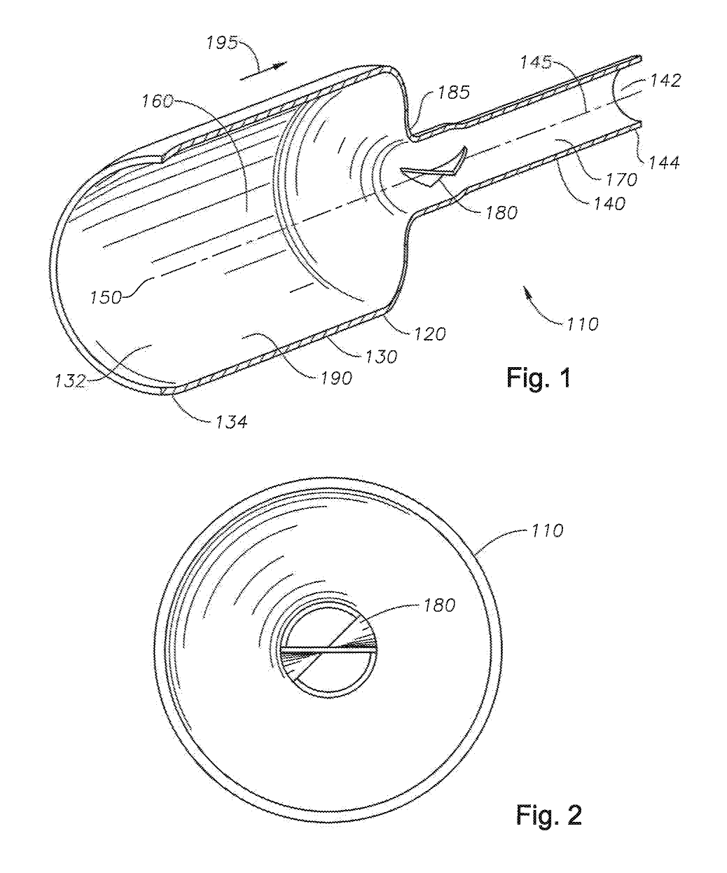

FIGS. 1 and 2 show an example cannula 110 for generating a spiral, laminar flow according to some implementations. The cannula 110 includes a body 120 having a first portion 130 and a second portion 140. A first opening 132 is provided at a first end 134, and a second opening 142 is provided at a second end 144. A longitudinal axis 145 may extend along an axial length of the cannula 110. In some instances, the first portion 130 and the second portion 140 have a generally cylindrical shape. Also, the first portion 130 may have a larger diameter than the second portion 140. A body 120 defines a passageway 150 formed form a first bore 160 defined by the first portion 130 and a second bore 170 defined by the second portion 140. While FIG. 1 shows example a passageway 150 with different sizes bores 160 and 170, it is within the scope of the disclosure that the passageway 150 may have any number of differently sized bores. For example, the passageway 150 may have a plurality of bores that progressively decrease in size (e.g., diameter) along a direction of fluid flow. In other instances, the passageway 150 may include bores having different sizes (e.g., diameters) in other configurations.

A flow-altering device may be disposed along the passageway 150. For example, the example cannula 110 shown in FIG. 1 may include one or more blades 180 is disposed in the passageway 150 proximate to junction 185 of the first bore 160 and the second bore 170. While FIG. 1 shows a single blade 180, other implementations may use a plurality of blades 180. The one or more blades 180 may be coupled at one or more locations to an interior wall 190 of the passageway 150.

Each blade 180 may include a small angle of attack relative to the direction of fluid flow through the passageway 150. In some instances, the angle of attack (interchangeably referred to as “pitch”) of the blade 180 may be constant along a length of the blade 150 parallel with longitudinal axis 145 (“longitudinal length”). In some instances, the angle of attack of blade 180 may increase along the longitudinal length of the blade 150 in the direction of fluid flow. Consequently, the one or more blades 180 are operable to generate a spiral fluid flow within the laminar flow regime. The angles of attack of the blade 180 and the rate at which the angle of attack may change along the longitudinal length of the blade 180 may be selected to control the generation of the laminar, spiral flow.

In some instances, the cannula 110 may include a plurality of blades 180 and a location of one or more of the plurality of blades 180 may be longitudinally staggered along axis 180 from one or more other blades 180. Further, in some instances, one or more blades 180 may be radially offset from one or more other blades 180. In other instances, the cannula 110 may include a plurality of blades 180 in which one or more blades 180 is both longitudinally and radially offset from one or more other blades 180. In some implementations, one or more of the blades 180 may have a constant angle of attack relative to the fluid flow along the longitudinal length of the blade 180. In some instances, the cannula 110 may include one or more blades 180 having different angles of attack and/or one or more blades 180 with a constant angle of attack and/or one or more other blades 180 having a variable angle of attack along a longitudinal length thereof.

As shown in greater detail in FIG. 2, in some instances, an example blade 180 extends diametrically across the passage 150 of the cannula 110. Further, the blade 180 includes a twist along a length thereof. As explained above, the twist of the blade 180 may be such that the an upstream edge of the blade 180 may have a low angle of attack relative to the fluid flow, while a downstream edge of the blade 180 may have a larger angle of attack relative to the fluid flow. While a single blade 180 is shown in FIG. 2, a plurality of blade 180 may be included along a length of the cannula 110. For example, one or more blades 180 may be disposed in the first bore 160, one or more blade 180 may be disposed in the second bore 170, one or more blades 180 may be disposed in the portion of the cannula 110 at the junction 185 of the first and second bores 160, 170, or a plurality of blades 180 at one or more of these locations may be disposed in an example cannula 110.

Additionally, while FIG. 2 shows blade 180 coupled to the interior wall 190 at two locations, in other instances, one or more blades 180 of the cannula 110 may be cantilevered. For example, one or more blades 180 may be coupled at only a single location to the interior wall 190. The one or more blades 180 may extend sufficiently from the interior wall 190 to not only induce a spiral flow of the fluid within the passageway 150 near the interior wall 190 but also fluid within the passageway 150 distant from the interior wall 190, e.g., fluid near the central portion of the passageway 150. Thus, in such implementations, the one or more blades 180 may efficiently generate the spiral flow since fluid moving within the passageway 150 away from the interior wall 150 (e.g., near a center of the passageway 150) tends to have a higher velocity than the fluid near the interior wall 150.

The angle of attack of the blades (either constant or variable) may be selected based on numerous factors. For example, the angle of attack may be selected based on the flow rate of the fluid through the cannula 110, a viscosity of the fluid, the geometry of the cannula, as well as other factors. Also, while FIG. 1 shows a blade 180 disposed in the second bore 170, in other implementations, one or more blades 180 may be disposed in the first bore 160 without any blades 80 disposed in the second bore 170. In still other instances, the cannula 110 may include one or more blades 180 disposed in the first bore 160 in conjunction with one or more blades 180 disposed in the second bore 170.

For the example cannulas 110 shown in FIGS. 1 and 2, in operation, fluid may enter the cannula 10 at the first opening 132, pass through the passageway 150 in the direction of arrow 195. The fluid accelerates as it transitions from the first bore 160 to the second 170. The accelerating fluid flows over the one or more blades 180, generating a spiral flow profile. Also, in some instances, one or more blades may be disposed in the first bore 160 such that spiral flow is generated as the fluid passes through the first bore 160. Further, the geometry of the one or more blades 180 is such that the flow of the fluid remains laminar. Consequently, a pressure drop across the cannulas 110 may be minimized thereby providing a larger flow rate through the cannulas 110. The fluid flow exits the cannula 110 through the second opening 142. The fluid exiting from the cannula 110 is a radially diffuse flow.

An important aspect of this diffuse flow exiting the example cannula 110 shown in FIG. 1 as well as the other example cannulas described herein is that the jet of fluid exiting the cannula 110 is avoided. A jet of fluid exiting a cannula disposed in a posterior segment of the eye may impinge upon the retina within the eye. This jet of fluid may agitate the retina. For retinas including a macular hole, the jet of fluid may pass through the macular hole, between the interior wall of the eye and the retina, and dislodge all or a part of the retina. Therefore, not only do the cannulas described herein provide for laminar flow through the cannula, reducing a pressure drop therethrough and having a higher flow rate as compared to turbulent flow, the fluid flow exiting the cannulas is diffuse reducing and/or avoiding potential agitation and/or injury to tissues of the body.

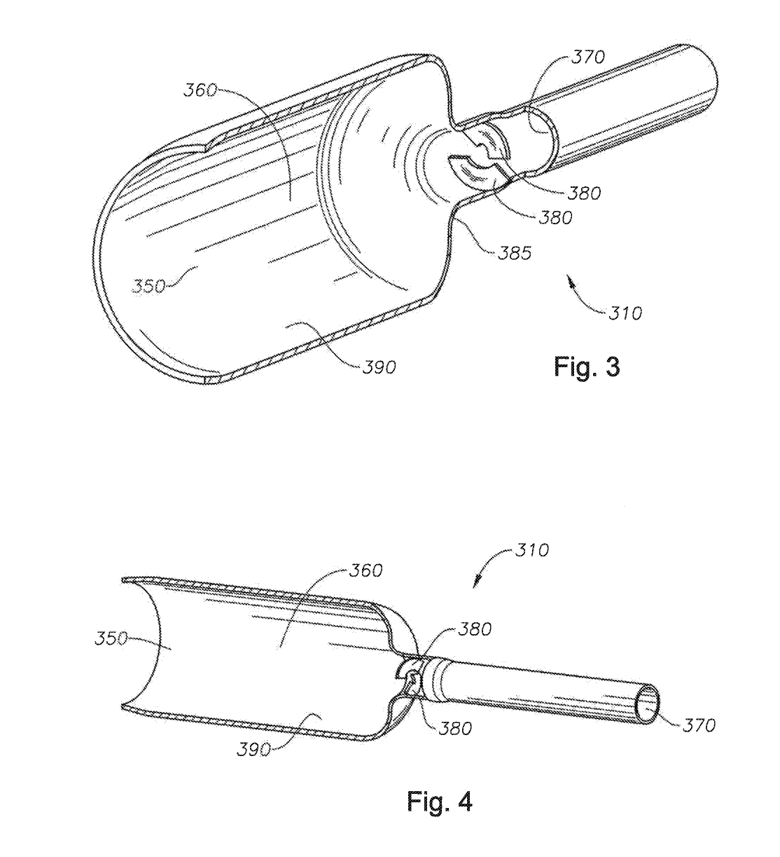

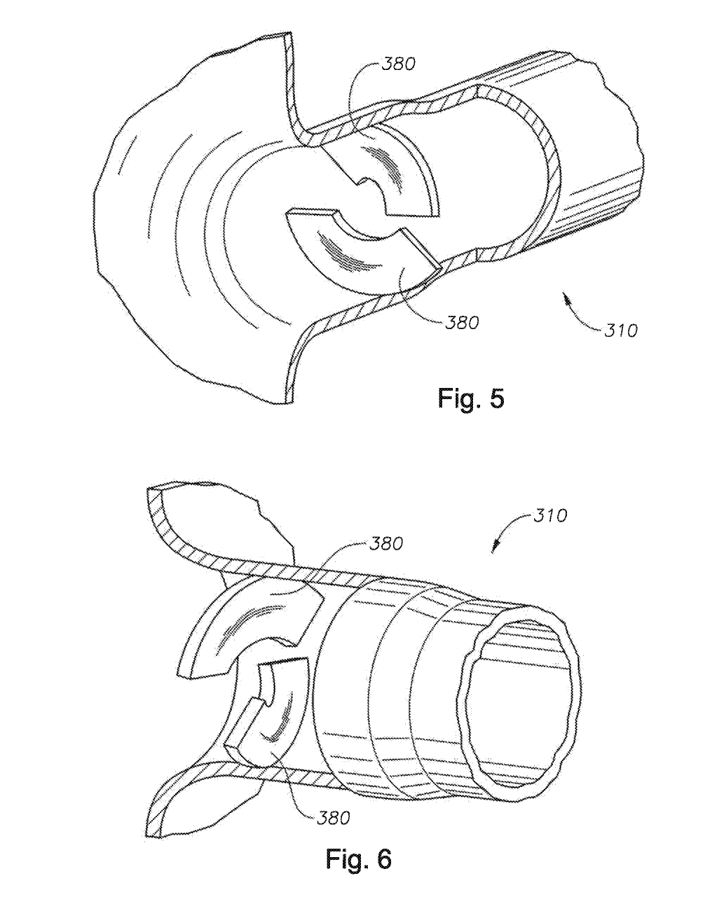

FIGS. 3-6 show another example cannula 310 similar to the cannula 110. As shown in FIG. 3, a flow-altering device in the form of baffles 380 may be included in the passageway 350 of the cannula 310. As shown, a pair of baffles 380 are disposed in a portion of second bore 370 adjacent to an interface between first bore 360 and second bore 370. In other instances, more or fewer baffles may be disposed in the passageway 350. For example, in some implementations, the cannula 310 may include a single baffle 380, while, in others, the cannula 310 may include two, three, four, or any number of desired baffles 380. Further, one or more baffles 380 may be disposed in the first bore 360. Thus, in some instances, one or more baffles 380 may occupy both the first bore 360 and the second bore 370. Further, one or more baffles 380 may extend from the first bore 360 to the second bore 370.

The baffle 380 may be in the form of an arc-shaped member radially extending from an interior wall 390 of the cannula 310 into the passage 350. The baffle 380 may also have a helical shape such that a baffle 380 extends along a longitudinal distance of the cannula 310. In some instances, the baffle 380 may radially extend along the interior wall 290 approximately 90°. In other instances, the baffle 380 may have a greater or smaller arc length. For example, in some instances, the baffle 380 may have an arc length less than or greater than 90°. Further, in some instances, baffles 380 may be disposed at a same position along a longitudinal length of the cannula 310. In other instances, baffles 380 may be disposed at different locations along the length of the cannula 310. For example, in some instances, two or more baffles 380 may overlap each other by at least a portion thereof. In other instances, a baffle 380 may not longitudinally overlap one or more other baffles 380. The one or more baffles 380 are operable to generate a spiral flow of the fluid passing through the cannula 310 while maintaining the fluid flow in the laminar flow regime.

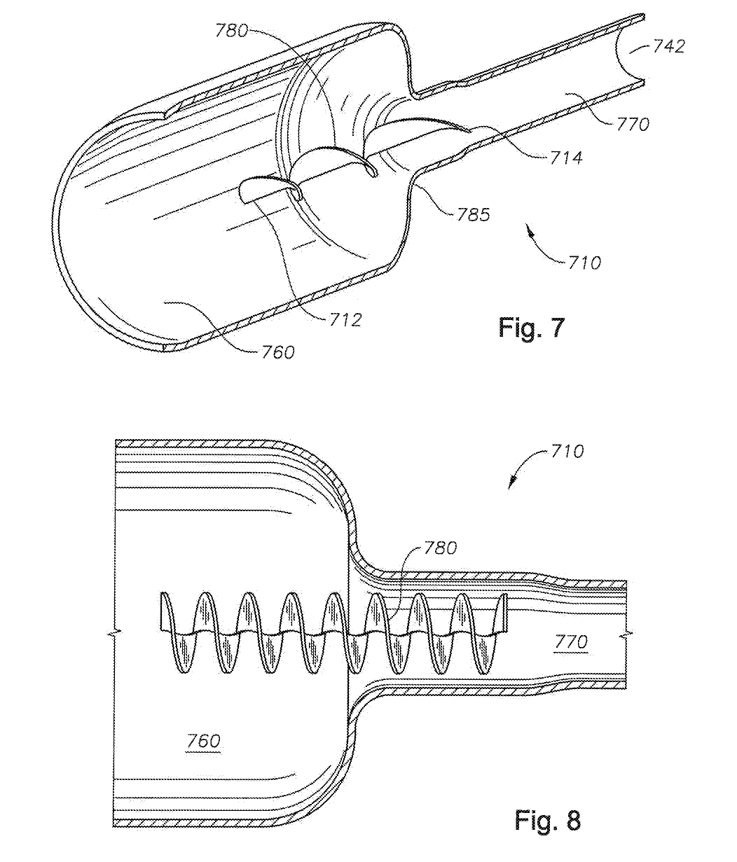

FIG. 7 illustrates another example cannula 710 similar to the cannulas 110 and 310, described above. However, the cannula 710 includes a flow-altering device in the form of a spiral member 780. In some instances, as shown in FIG. 7, a portion of the spiral member 780 may extend into the first bore 760, and a portion of the spiral member 780 may extend into the second bore 770. In other implementations, the spiral member 780 may reside exclusively in either of the first bore 760 or the second bore 770. Still further, a plurality of spiral members 780 may reside in passageway 750. For example, one or more spiral members 780 may exclusively reside in the first bore 760, and one or more spiral members 780 may reside in the second passage 770.

Additionally, the spiral member 780 may be coupled to an interior wall 790 of passageway 750. For example, the spiral member 780 may be coupled at one or more locations along the length of the spiral member 780. For example, for a spiral member 780 extending into both the first bore 760 and the second bore 770, the spiral member 780 may be coupled to the interior wall 750 at one or more locations in the first bore 760 and at one or more locations within the second bore 770. In other instances, the spiral member 780 may be coupled to the interior wall 750 at one or more locations exclusively in the first bore 760 or the second bore 770. In still other instances, the spiral member 780 may be coupled to the interior wall 790 along an entire length of the spiral member 780. Still further, the spiral member 770 may be coupled to the interior wall 750 at junction 785 between the first bore 760 and the second bore 770.

In some implementations, as shown in FIG. 7, a pitch of the spiral member 780 may decrease from a point upstream in the cannula 710 to a location downstream in the cannula 710. That is, in some instances, the spiral member 780 may have a higher angle of attack relative to the fluid flow at a first end 712 (an upstream position) and a shallower angle of attack at a second end 714 (a downstream position). In other instances, the angle of attach of the spiral member 780 may progressively increase from a shallow angle of attack at the first end 712 to a larger angle of attack at the second end 714.

In some instances, the angle of attack of the spiral member 780 may change gradually along a length of the spiral member 780. In some implementations, the angle of attack may change linearly along the length of the spiral member 780, while, in other instances, the angle of attack may change nonlinearly along the length of the spiral member 780. In other implementations, the angle of attack of the spiral member 780 may change linearly along one or more portions of its length and non-linearly along one or more other portions of its length.

In other instances, the spiral member 780 may have a small angle of attack at the first end 712 and a larger angle of attack at the second end 714. Alternately, the angle of attack of the spiral member 780 may increase along only a portion thereof. In still other instances, the angle of attack of the spiral member 780 may progressively increase along portions of the spiral member 780 while other portions of the spiral member 780 may have a constant pitch. Still further, the angle of attack may change linearly or nonlinearly along one or more portions of the length of the spiral member 780. In some instances, the angle of attack of the spiral member 780 may increase linearly along one or more portions, non-linearly along one or more other portions, and, in some instances, include a portion that has a constant angle of attack.

FIG. 8 shows another implementation of cannula 710 in which the spiral member 780 has a constant pitch along an entire length thereof. As shown, the spiral member 780 extends through a portion of both the first bore 760 and the second bore 770. As explained above, the spiral member 780 may reside solely the first bore 760 or the second bore 770.

The angle of attack of the spiral member 780 (whether constant or variable over its length) is selected so as to maintain flow in the laminar flow regime. Thus, the pitch of the spiral member 780 may be selected based on one or more factors, such as one or more of the factors described above. Accordingly, the fluid passing through the cannula 710 is formed into a spiral, laminar flow by the spiral member 780 forming a diffuse fluid flow exiting second opening 742. The diffuse flow may significantly reduce or eliminate agitation and/or injury to tissues by avoiding the creation of a jet of fluid exiting the cannula 710.

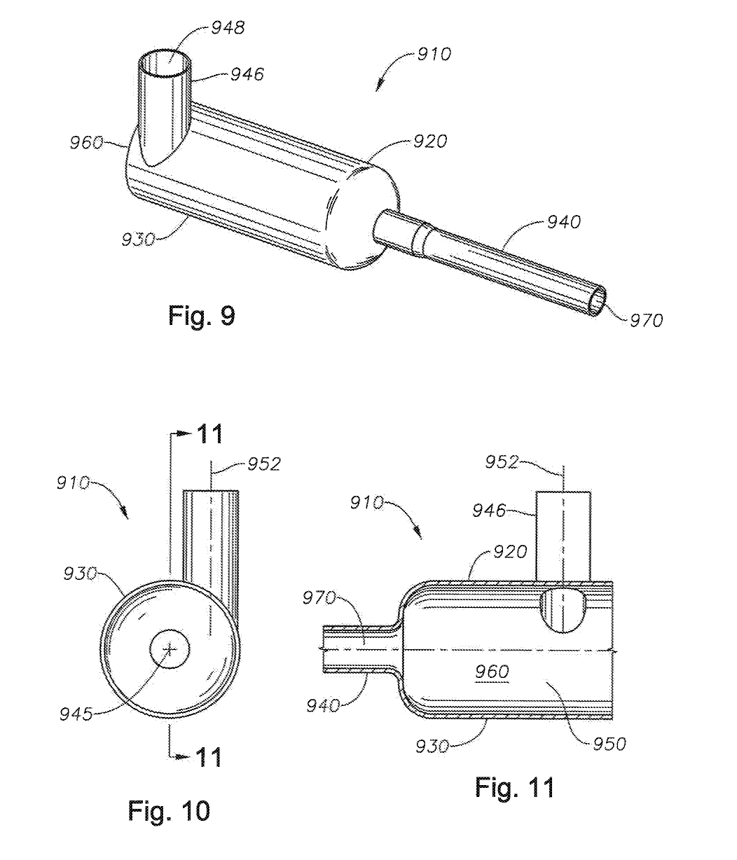

FIGS. 9-17 illustrate additional implementations of cannulas for generating spiral, laminar flow. FIG. 9 is a perspective view of an example cannula 910. Cannula 910 may similar to one or more of the other cannulas described herein. For example, cannula 910 may include a body 920 defining a first portion 930 and a second portion 940. The first portion 930 defines a first bore 960, and the second portion 940 defines a second bore 970. The cannula 910 may also include a flow tube 946 extending into the first portion 930 such that a bore 948 defined by the flow tube 946 is in communication with the first bore 960.

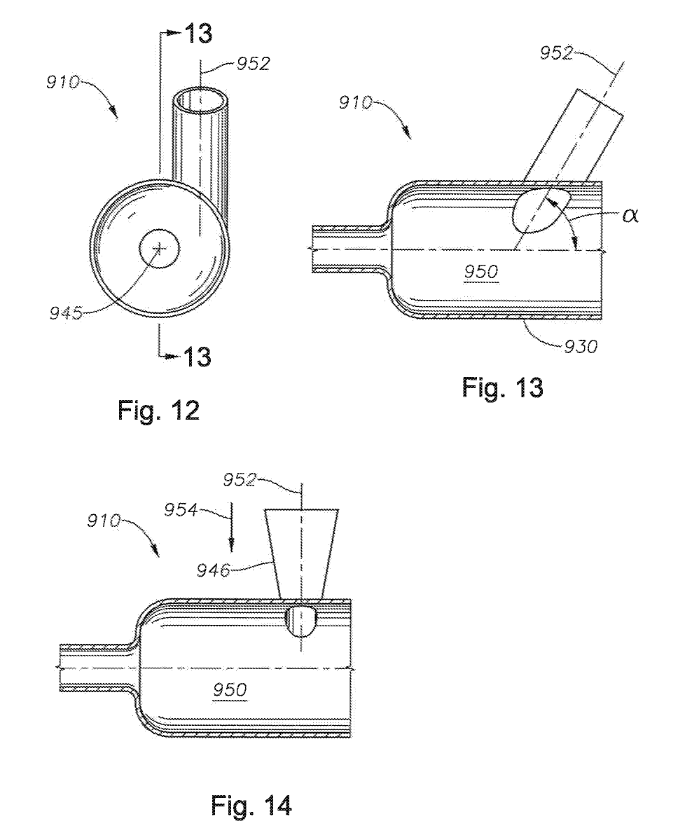

In some implementations, such as the example cannulas shown in FIGS. 10 and 11, a centerline (interchangeably referred to as “longitudinal axis”) 952 of the flow tube 946 is perpendicular to longitudinal axis 945 of the cannula 910. Alternately, as shown in FIGS. 12 and 13, the centerline 952 of the flow tube 946 may be oblique to the longitudinal axis 945 of the cannula 910. As shown particularly in FIG. 13, the centerline 952 is at an angle, α, relative to the longitudinal axis 945 is acute. In other instances, the angle, α, may be obtuse.

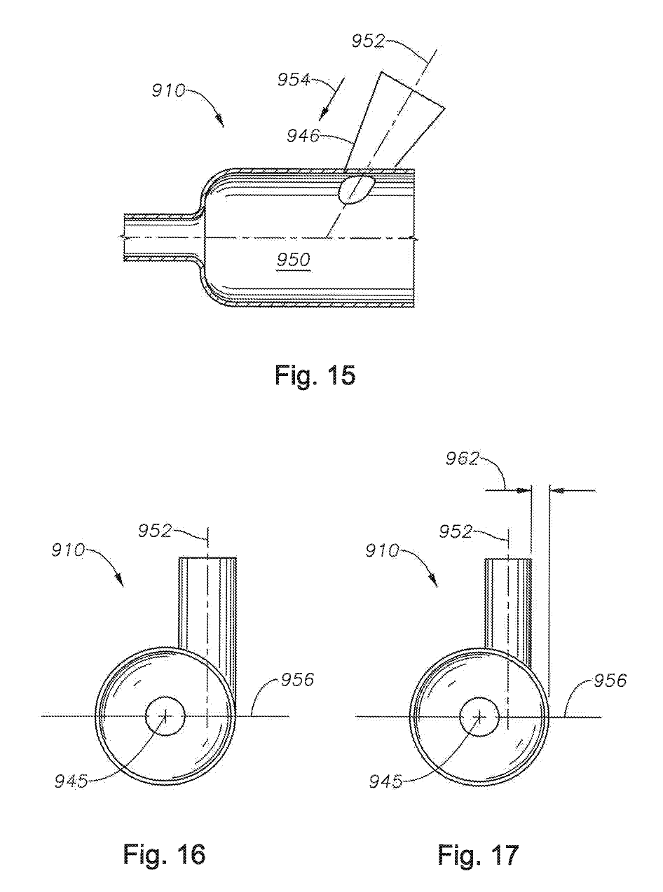

In FIGS. 10-13, the flow tube 946 is shown as a hollow, cylindrical member. However, the flow tube 946 may have other forms. For example, as shown in FIGS. 14 and 15, the flow tube 946 may include a tapered form. As shown, the taper of flow tube 946 has a decreasing cross-section (such as by a decreasing diameter) along the flow direction, indicated by arrow 954. In other instances, the flow tube 946 may include a taper in which the cross section (or diameter) increases along the flow direction of arrow 954. However, the shape of the flow tube 946 is not limited to a cylindrical or tapered shape, but may have any suitable shape.

In some instances, as shown in FIG. 16, the flow tube 946 may intersect the first portion 930 such that an outer surface of the flow tube 946 is flush with an outer surface of the first portion 930 at a location where a radial line 956 of the first portion 930 extends through the longitudinal axis 945. In other implementations, a distance 962 may exist between the outer surface of the flow tube 946 and the outer surface of the first portion 930. Still further, a size (e.g., a diameter) of the flow tube 946 may be equal to or smaller than the radius of the first bore 960. In other instances, the size (e.g., diameter) of the flow tube 946 may be larger than the radius of the first bore 960. For example, in some instances, the size (e.g., diameter) of the flow tube 946 may be equal to or larger than the size (e.g., diameter) of the bore 960. While the sizes of the flow tube 946 and the first bore 960 is described in terms of diameter and/or radius, the cross-sectional shapes of the flow tube 946 and the first bore 960 may be non-circular along their entire respective lengths or one or more portions thereof.

Further, in regards to one or more of the cannulas described herein, the first bore and the second bore is described as being substantially cylindrical, the bores are not so limited. That is, the first bore and/or the second bore may have a non-cylindrical shape. For example, the first bore and/or the second bore may have a tapered shape.

Referring again to the example cannulas shown in FIGS. 9-17, in some implementations, a first flow may be passed through the passageway 950 and a second flow may be passed through the flow tube 946. The two flows interact to generate a spiral, laminar flow. In other instances, the first flow through the passageway 950 may be eliminated. For example, in some instances, a first opening 932 may be sealed. Thus, the second flow through the flow tube 946 may be introduced into the passageway 950 to form a laminar, spiral flow that exits through a second opening 942 as a diffuse flow.

In some instances, the cannulas described herein may be a 23 gauge, 25 gauge, or 27 gauge cannulas. In still other implementations, one or more of the cannulas described herein may have a larger or smaller gauge sizes. Further, the cannulas may be adapted for use in ophthalmic surgical procedures. However, the cannulas may be used for other surgical procedures, particularly surgical procedures involving the infusion of fluids close to delicate or sensitive tissues.

It should be understood that, although many aspects have been described herein, some implementations may include all of the features, others may include some features while including other, different features, and in still other instances, other implementations may omit some features while including others. That is, various implementations may include one, some, or all of the features described herein.

A number of implementations have been described. Nevertheless, it will be understood that various modifications may be made without departing from the spirit and scope of the disclosure. Accordingly, other implementations are within the scope of the following claims.

What is claimed is:

1. An infusion cannula comprising:
a body comprising:
a first portion defining a first bore; and
a second portion defining a second bore, the first bore and the second bore in fluid communication with each other;
a passageway defined by the first bore and the second bore, the passageway extending through the body; and
a flow-altering device adapted to generate a spiral, laminar flow of fluid passing through the passageway.

2. The infusion cannula of claim 1, wherein the first bore has a cross-sectional area that is larger than a cross-sectional area of the second bore.

3. The infusion cannula of claim 1, wherein the first bore and the second bore have circular cross-sections and wherein the first bore has a larger cross-sectional area than the second bore.

4. The infusion cannula of claim 1, wherein the flow-altering device comprises a blade disposed at a location in the passageway.

5. The infusion cannula of claim 4, wherein the blade is disposed in the second bore proximate a junction between the first bore and the second bore.

6. The infusion cannula of claim 5, wherein the blade comprises a leading edge at an upstream location and a trailing edge at a downstream location and wherein an angle of attack of the leading edge is less than an angle attack of the trailing edge.

7. The infusion cannula of claim 4, wherein the blade is coupled to an interior wall of the passageway at least one location.

8. The infusion cannula of claim 7, wherein the blade is coupled to the interior wall of the passageway at two locations.

9. The infusion cannula of claim 4, wherein the blade diametrically extends across the passageway.

10. The infusion cannula of claim 4, wherein a plurality of blades are disposed in the passageway.

11. The infusion cannula of claim 10, wherein a first portion of the plurality of blades is disposed in the first bore and a second portion of the plurality of blades is disposed in the second bore.

12. The infusion cannula of claim 10, wherein at least one blade is radially offset from another blade.

13. The infusion cannula of claim 1, wherein the flow-altering device is a spiral member disposed at a location along the passageway.

14. The infusion cannula of claim 13, wherein the spiral member comprises a leading edge at an upstream location and a trailing edge at a downstream location, the leading edge having a first angle of attack and the trailing edge having a second angle of attack.

15. The infusion cannula of claim 14, wherein the first angle of attack is greater than the second angle of attack.

16. The infusion cannula of claim 14, wherein an angle of attack of the spiral member varies from the first angle of attack to the second angle of attack.

17. The infusion cannula of claim 13, wherein an angle of attack of the spiral member decreases from a first angle of attack at a leading edge of the spiral member at an upstream location to a second angle of attack at a trailing edge of the spiral member at a downstream location.

18. The infusion cannula of claim 13, wherein an angle of attack of the spiral member increases from a first angle of attack at a leading edge of the spiral member at an upstream location to a second angle of attack at a trailing edge of the spiral member at a downstream location.

19. The infusion cannula of claim 13, wherein the spiral member has a constant angle of attack along a length thereof.

20. The infusion cannula of claim 13, wherein the spiral member extends through a portion of the first bore and through a portion of the second bore.
